(19) United States
(12) Patent Application Publication
(10) Pub. No.: US 2026/0272971 A1
(43) Pub. Date: Sep. 17, 2026

(54) ISOLATED MITOCHONDRIA AS DELIVERY VEHICLES AND STIMULATORS OF PROTEIN SYNTHESIS

(71) Applicant: MINOVIA THERAPEUTICS LTD, Tirat Hacarmel (IL)

(72) Inventors: Natalie YIVGI-OHANA, Haifa (IL); Noa SHER, Haifa (IL)

(21) Appl. No.: 19/669,854

(22) Filed: May 6, 2026

Related U.S. Application Data
(63) Continuation of application No. PCT/IL2024/051129, filed on Nov. 27, 2024.
(60) Provisional application No. 63/604,061, filed on Nov. 29, 2023.

(51) Int. Cl. A61K 31/711; A61K 38/00; A61P 35/00; C12N 9/02; C12N 9/10; C12N 9/12; C12N 15/62

(52) U.S. Cl. CPC A61K 31/711; A61P 35/00; C12N 9/0008; C12N 9/0051; C12N 9/1029; C12N 9/1252; C12N 15/62; A61K 38/00; C07K 2319/07; C12Y 102/04004; C12Y 108/01004; C12Y 203/01168; C12Y 207/07007

(57) ABSTRACT

The present invention relates generally to isolated mitochondria derived from mtDNA-depleted cells (Rho0-mito) that are advantageously used as delivery vehicles for heterogenous proteins or nucleic acids or chemical agents. In some embodiments, proteins or nucleic acids or chemical agents are introduced into a cell, transported into, transferred or expressed in the mitochondria, thereafter or before mtDNA is removed and mtDNA-free mitochondria are extracted from the cell. These mitochondria are used for therapeutic treatment of mitochondria-related diseases such as maple syrup syndrome, citrin deficiency, Nieman-Pick disease, X-linked adrenoleukodystrophy (X-ALD), Pyruvate Dehydrogenase Deficiency, respiratory chain diseases, mutations in mitochondrial respiratory chain proteins, fatty acid metabolism disorders, or cancer with reduced likelihood of causing an immunogenic response. Additionally, in some embodiments the present invention relates to isolated mitochondria, including but not necessarily isolated mitochondria derived from mtDNA-depleted cells (Rho0-mito), that are exogenously introduced into cells to enhance production of endogenous or heterogenous proteins.

### CROSS REFERENCE TO RELATED APPLICATIONS

This application is a Bypass Continuation of PCT/IL2024/051129 having International filing date of Nov. 27, 2024, which claims the benefit of priority of U.S. Provisional Patent Application No. 63/604,061, filed Nov. 29, 2023, the contents of which are all incorporated herein by reference in their entirety

### FIELD OF THE INVENTION

The present invention relates generally to isolated mitochondria and more specifically to isolated mitochondria depleted of mitochondrial DNA (mtDNA)(Rho0-mitochondria) for use as delivery vehicles or treatment of mitochondrial and metabolic diseases, obesity or cancer, or for improved production of protein in cells.

### BACKGROUND

Many diseases are caused by dysfunctional enzymes or proteins. Some of these, such as mitochondrial disorders or lysosomal storage disorders, may be treatable by enzyme replacement therapy like approaches, but delivery of DNA, RNA or enzyme to the mitochondria is a challenge. It is possible to inject isolated mitochondria systemically or locally, but there are concerns that mitochondrial therapies may cause an immune response against allogeneic mtDNA.

Therapeutic proteins such as antibodies or vaccines for the pharmaceutical industry, as well as proteins for the food industries such as enzymes or peptides, are of high industrial demand.

Cell production of such industrial proteins commonly utilizes, for example, CHO cells mainly in the pharmaceutical industry, or HEK293 cells that are frequently used by the gene therapy industry to produce viruses. The food industry uses cultured cells to produce commercially valuable proteins such as whey and casein, also produced in yeast cells. Cultured meat requires cells with high protein content. Cultured plant cells are used as well, for example, for collagen synthesis.

There is, therefore, a need for mitochondrial therapies that deliver therapeutic enzymes or proteins with reduced risk of provoking an immune response, and for improving the efficiency and yield of production of such proteins, and other, industrially made using cultured cells.

### SUMMARY OF THE INVENTION

There According to some aspects, the present invention provides methods for increasing protein synthesis in a cell by contacting a cell with isolated exogenous mitochondria, the isolated exogenous mitochondria either contain their mitochondrial DNA (mtDNA) or substantially depleted of their mtDNA (Rho0-mito), and the cell includes any mammalian cell, a plant cell, or a yeast cell commonly used for industrial protein production.

Surprisingly, in some embodiment, the cell or population of cells comprising the isolated exogenous mitochondria were herein exemplified to have improved capability to synthesize endogenous proteins or heterologous proteins, as well as enhanced proliferation rate, the former is herein demonstrated as increased rate/level of protein synthesis, increased intracellular ATP concentrations, and elevated ability to consume branched chain amino acids (BCAA).

According to another aspect, the present invention provides an advantageous isolated mitochondria substantially free of mitochondrial DNA (mtDNA) (Rho0-mito). In some embodiments, the Rho0-mito contains heterologous mitochondrially targeted protein or nucleic acid sequence encoding the heterologous protein, or a chemical agent.

Further advantageous is the seminal discovery that mitochondria derived from mtDNA-depleted cells can be isolated (isolated Rho0) and used fresh or cryopreserved to deliver cargo in vivo to cells of a subject in need with reduced likelihood of immunogenicity, in some embodiments.

In a related aspect, the present invention provides methods of treatment of a disease or disorder using Rho0-mito. The Rho0-mito, or compositions comprising the same, are administered at an effective amount to a subject in need of therapeutic treatment for mitochondria-related diseases, metabolic disease, obesity or cancer, and advantageously, in some embodiments, comprise as a cargo for delivery into the subject's cells a heterologous protein or nucleic acid sequence encoding such an heterologous protein, or a chemical agent that have the desired therapeutic effect.

In yet another aspect, also provided herein is a method for producing the isolated mitochondria substantially free of mitochondrial DNA (mtDNA) (Rho0-mito), including the steps of removing mitochondrial DNA from the mitochondria of a cell and isolating the mitochondria from the cell, wherein in some embodiments, the method also include a step of contacting the cell with a chemical agent or a nucleic acid encoding a heterologous mitochondrially targeted protein prior to or following the removal of mitochondrial DNA from the mitochondria, and before isolating the mitochondria from the cell.

In one aspect, the present disclosure provided herein relates to isolated mitochondria substantially free of mitochondrial DNA (mtDNA) (Rho0-mito). In some embodiments, the Rho0-mito comprises a heterologous protein or nucleic acid sequence. In some embodiments, the Rho0-mito comprises a chemical agent. According to one aspect, there is provided an isolated mitochondria substantially free of mitochondrial DNA (Rho0-mito), the Rho0-mito comprises a heterologous protein or nucleic acid sequence and/or a chemical agent. Each possibility is a separate embodiment.

In one aspect, the heterologous nucleic acid sequence is single stranded or double stranded. Each possibility is a separate embodiment. In certain aspects, the nucleic acid sequence is in an expression vector. In various aspects, the heterologous protein or nucleic acid sequence includes a mitochondrial-targeting sequence. Each possibility is a separate embodiment. In one aspect, the nucleic acid sequence is DNA or RNA. In a further aspect, the protein comprises a peptide. In some aspects, the protein comprises an enzyme. In some embodiments, the protein is a peptide, a polypeptide, an enzyme, a transcription factor, an antibody or binding fragment thereof. Each possibility is a separate embodiment.

In some embodiments, the nucleic acid encodes one or more of: BCKDK, SLC25A13, SMPD1, ABCD1, SerpinA1, SerpinA3, CPT1A, SLC25A20, PDHA1, ACADVL, HADHA, ACADM, and ACADS, or any combination thereof. Each possibility is a separate embodiment.

In some embodiments, the protein comprises one or more proteins related to enzymatic deficiencies. In some embodiments, the protein comprises one or more of a branched-chain α-ketoacid dehydrogenase complex, citrin, acid sphingomyelinase, ATP binding cassette subfamily D member 1, alpha-1 antitrypsin, alpha 1-antichymotrypsin, carnitine palmitoyltransferase 1A, carnitine-acylcarnitine translocase, very long-chain specific acyl-CoA dehydrogenase, mitochondrial trifunctional protein, acyl-coenzyme A dehydrogenase, and short-chain acyl-CoA dehydrogenase, or any combination thereof. Each possibility is a separate embodiment.

In some embodiments, the chemical agent comprises one or more of: FCCP, Chlorfenapyr, Pinacyanol, Oligomycin, 2,4-dinitrophenol, Coenzyme Q10, alpha lipoic acid, and resveratrol, or any combination thereof. Each possibility is a separate embodiment.

In some embodiments, the isolated mitochondria comprise cryogenically preserved mitochondria.

In some aspects, the present disclosure relates to an isolated cell or population of cells comprising an isolated exogenous mitochondria substantially free of mitochondria DNA (mtDNA) (exogenous Rho0-mito), according to any one of the preceding embossments. In some aspects, the present disclosure relates to a cell, or population of cells, comprising an isolated exogenous mitochondria substantially free of mitochondria DNA (mtDNA) (exogenous Rho0-mito), the exogenous Rho0-mito comprises a heterologous protein or nucleic acid sequence. Each possibility is a separate embodiment. In some aspects, the present disclosure relates to a cell comprising an isolated exogenous mitochondria substantially free of mitochondria DNA (mtDNA) (exogenous Rho0-mito), the exogenous Rho0-mito comprises a chemical agent. In some aspects, the present disclosure relates to a cell comprising an isolated exogenous mitochondria substantially free of mitochondria DNA (mtDNA) (exogenous Rho0-mito), the exogenous Rho0-mito comprises a heterologous protein or nucleic acid sequence and/or a chemical agent. Each possibility is a separate embodiment.

In some embodiments, the cell or population of cells comprising the isolated exogenous Rho0-mito can synthesize a protein at an improved synthesis rate/level, and wherein the protein comprises an endogenous protein or a heterologous protein. Each possibility is a separate embodiment.

In some embodiments, the improved synthesis rate/level comprises an increase of least 1.2×, at least 1.4×, at least 1.5×, at least 1.6×, at least 1.8×, at least 2×, at least 2.5×, at least 3×, at least 4×, or at least 5×, or more, compared to the synthesis rate/level of a cell not comprising isolated exogenous mitochondria. Each possibility is a separate embodiment.

In some embodiments, the cell comprising the isolated exogenous Rho0-mito is capable of proliferating at a rate at least 1.2×, at least 1.4×, at least 1.5×, at least 1.6×, at least 1.8×, at least 2×, or faster compared to a cell not comprising exogenous mitochondria. Each possibility is a separate embodiment.

In some embodiments, the cell comprises a mammalian cell, a plant cell, or yeast cell. Each possibility is a separate embodiment.

In some embodiments, the cell comprises a Chinese hamster ovary (CHO) cell, a CHO-GFP cell, a CHO Agarabi cell, a HeLa cell or a HEK293 cell. Each possibility is a separate embodiment.

In some embodiments, the cell comprising the isolated exogenous Rho0-mito, wherein the isolated mitochondria is cryogenically preserved.

In one embodiment, the present disclosure provided herein relates to a method for producing isolated mitochondria substantially free of mitochondrial DNA (mtDNA) (Rho0-mito), which in some embodiments can be used for transplantation to subjects in need of functional mitochondria or in some instances can be further modified to provide a heterologous protein or nucleic acid sequence to a subject in need thereof. In some embodiments, such methods comprise: a) treating a cell to delete the mtDNA; b) removing mitochondrial DNA from the mitochondria; and c) isolating the mtDNA-depleted mitochondria (Rho0-mito) from the cell. In addition, in some embodiments, in any stage/step before isolation of mitochondria, cells harboring the mitochondria can be modified to contain a heterologous protein or nucleic acid sequence of a protein targeted to the mitochondria (e.g., an enzyme for enzyme replacement therapy or other therapeutic protein). In addition, in some embodiments, in any stage/step before isolation of mitochondria, cells harboring the mitochondria can be modified to contain a chemical agent that locates (transfers to) the mitochondria, the chemical agent has therapeutic benefits (e.g. anti-cancerous activity).

According to some aspects, there is provided a method for producing isolated mitochondria substantially free of mitochondrial DNA (mtDNA)(isolated Rho0-mito), the method comprises: a) removing mitochondrial DNA (mtDNA) from the mitochondria; and b) isolating the mitochondria (Rho0-mito) from the cell. In some embodiments, the step of removing the mtDNA from the mitochondria comprises treating the cells for deletion/depletion/degradation/clearance of the mtDNA.

In some embodiments, the method for producing isolated mitochondria substantially free of mitochondrial DNA (mtDNA)(isolated Rho0-mito) further comprising a step of contacting the cell with a chemical agent, active molecule, or a nucleic acid encoding a heterologous mitochondrially targeted protein prior to or following the removal of mitochondrial DNA from the mitochondria, and before isolating the mitochondria from the cell. Each possibility is a separate embodiment.

In some embodiments, the heterologous nucleic acid encodes one or more of: BCKDK, SLC25A13, SMPD1, ABCD1, SerpinA1, SerpinA3, CPT1A, SLC25A20, PDHA1, ACADVL, HADHA, ACADM, ACADS, BCKDH, TFAM, POLG, TK, and DGUOK, or any combination thereof. Each possibility is a separate embodiment.

In some embodiments, the heterologous protein comprises a peptide, a polypeptide, an enzyme, a transcription factor, an antibody or binding fragment thereof. Each possibility is a separate embodiment. In some embodiments, the heterologous protein or active molecule comprise a small organic molecule, a peptide, a polypeptide, an enzyme, a transcription factor, an antibody or binding fragment thereof. Each possibility is a separate embodiment.

In some embodiments, the protein comprises one or more of a branched-chain α-ketoacid dehydrogenase complex, citrin, acid sphingomyelinase, ATP binding cassette subfamily D member 1, alpha-1 antitrypsin, alpha 1-antichymotrypsin, carnitine palmitoyltransferase 1A, carnitine-acylcarnitine translocase, very long-chain specific acyl-CoA dehydrogenase, mitochondrial trifunctional protein, acyl-coenzyme A dehydrogenase, and short-chain acyl-CoA dehydrogenase, or any combination thereof. Each possibility is a separate embodiment.

In some embodiments, the chemical agent is selected from one or more of FCCP, Chlorfenapyr, Pinacyanol, Oligomycin and 2,4-dinitrophenol, or any combination thereof. Each possibility is a separate embodiment. In some embodiments, the chemical agents transfer to the mitochondria. In some embodiments, the chemical agent has therapeutic effect such as, but not necessarily limited to, anti-cancerous activity.

In another embodiment, the techniques described herein relate to a method for treating a disease or disorder in a subject, comprising administering to the subject an effective amount of isolated mitochondria substantially free of mitochondrial DNA (mtDNA) or a composition comprising an isolated mitochondria substantially free of mitochondrial DNA (mtDNA), according to any one of the preceding embossments. Each possibility is a separate embodiment.

According to some aspects, there is provided a method for treating a disease or disorder in a subject, comprising administering to the subject an effective amount of isolated mitochondria substantially free of mitochondrial DNA (mtDNA)(isolated Rho0-mito) or a composition comprising the same, the isolated Rho0-mito comprises a heterologous protein or nucleic acid sequence encoding a heterologous protein or a chemical agent. Each possibility is a separate embodiment.

In some aspects, the disease or disorder is selected from one or more of maple syrup syndrome, citrin deficiency, Nieman-Pick disease, X-linked adrenoleukodystrophy (X-ALD), Pyruvate dehydrogenase deficiency, respiratory chain diseases, mutations in mitochondrial respiratory chain proteins, and fatty acid metabolism disorders, or any combination thereof. Each possibility is a separate embodiment. In some embodiments, the disease or disorder is further selected from obesity and/or cancer. Each possibility is a separate embodiment.

In one aspect, the isolated mitochondria substantially free of mitochondrial DNA (mtDNA) is cryogenically preserved following isolation.

According to some embodiments, there is provided a composition comprising a mitochondria substantially free of mitochondria DNA (mtDNA) (isolated Rho0-mito) and a pharmaceutically acceptable carrier, according to any one of the preceding embodiments. Each possibility is a separate embodiment.

According to some embodiments, there is provided a composition comprising a mitochondria substantially free of mitochondria DNA (mtDNA) (isolated Rho0-mito), the Rho0-mito comprises a heterologous protein or nucleic acid sequence encoding a heterologous protein, and a pharmaceutically acceptable carrier. Each possibility is a separate embodiment.

According to some embodiments, the isolated mitochondria substantially free of mitochondria DNA (mtDNA) (isolated Rho0-mito) or the composition comprising the same, for use in treatment of a disease or disorder in a subject in need. Each possibility is a separate embodiment.

In some embodiments, the disease or disorder is selected from one or more of maple syrup syndrome, citrin deficiency, Nieman-Pick disease, X-linked adrenoleukodystrophy (X-ALD), Pyruvate Dehydrogenase Deficiency, respiratory chain diseases, mutations in mitochondrial respiratory chain proteins, and fatty acid metabolism disorders, or any combination thereof. Each possibility is a separate embodiment. In some embodiments, the disease or disorder is further selected from obesity and/or cancer. Each possibility is a separate embodiment.

According to some aspects there is provided a method for increasing protein synthesis in a cell or population of cells, the method comprises: expressing a protein of interest in a cell in culture and contacting the cell with isolated exogenous mitochondria; thereby enhancing synthesis rate/level of a protein expressed in the cell containing the exogenous mitochondria.

In some embodiments, the isolated exogenous mitochondria is substantially free of mitochondrial DNA (mtDNA)(Rho0-mito).

In some embodiments, the protein of interest comprises an endogenous protein and/or a heterologous protein. Each possibility is a separate embodiment.

In some embodiments, the contacting of the cell with isolated exogenous mitochondria comprises incubating the cell in culture with 2.2 miliunits-17.6 miliunits CS activity of isolated exogenous mitochondria per 1.0E6 cells.

In some embodiments, the method further comprises isolating the protein of interest from the cells or culture.

In some embodiments, the cell comprises a mammalian cell, a plant cell, or a yeast cell, or any combination thereof.

In some embodiments, the cell comprises a Chinese hamster ovary (CHO) cell, a CHO-GFP cell, a CHO Agarabi cell, a HeLa cell or a HEK293 cell, or any combination thereof. Each possibility is a separate embodiment.

In some embodiments, the protein of interest comprises one or more of a therapeutic agent and/or a vaccine, a peptide, a polypeptide, an enzyme, a transcription factor, an antibody or binding fragment thereof. Each possibility is a separate embodiment. In some embodiments, the protein of interest comprises a therapeutic agent and/or a vaccine. Each possibility is a separate embodiment.

In some embodiments, the improved synthesis rate/level of the protein in the cell comprises an increase of least 1.2×, at least 1.4×, at least 1.5×, at least 1.6×, at least 1.8×, at least 2×, at least 2.5×, at least 3×, at least 4×, or at least 5×, or more, compared to the synthesis rate/level of the protein in a cell not comprising isolated exogenous mitochondria.

In some embodiments, the improved synthesis rate/level of the protein in the cell comprises an increase between 1.2× and 5×, or between 1.2× and 3×, compared to the synthesis rate/level of the protein in a cell not comprising isolated exogenous mitochondria. Each possibility is a separate embodiment.

In some embodiments, the cell can proliferate at a rate at least 1.2×, at least 1.4×, at least 1.5×, at least 1.6×, at least 1.8×, at least 2×, or faster compared to a cell not comprising isolated exogenous mitochondria.

In some embodiments, the improved synthesis rate/level of the protein in the cell comprises an increase in protein synthesis rate/level per intracellular ATP concentrations of least 1.2×, at least 1.4×, at least 1.5×, at least 1.6×, at least 1.8×, at least 2×, at least 2.5×, at least 3×, at least 4×, or at least 5×, or more, compared to the protein synthesis rate/level per intracellular ATP concentrations in a cell not comprising isolated exogenous mitochondria. In some embodiments, the increase in protein synthesis rate/level per intracellular ATP concentrations is between 1.2× and 5×, or between 1.2× and 3×, compared to the protein synthesis rate/level per intracellular ATP concentrations in a cell not comprising isolated exogenous mitochondria. Each possibility is a separate embodiment.

In some embodiments, the isolated exogenous mitochondria is cryogenically preserved prior to contacting the cell.

In some embodiments, the protein of interest comprises one or more of BCKDK, SLC25A13, SMPD1, ABCD1, SerpinA1, SerpinA3, CPT1A, SLC25A20, PDHA1, ACADVL, HADHA, ACADM, ACADS, BCKDH, TFAM, POLG, TK, and DGUOK, or any combination thereof. Each possibility is a separate embodiment.

According to yet another aspect, the present disclosure provides an isolated cell or population of cells comprising isolated exogenous mitochondria and overexpressing a protein of interest, wherein the cell or population of cells comprising the isolated exogenous can synthesize the protein of interest at an improved synthesis rate/level, compared to the synthesis rate/level of the protein in a cell not comprising isolated exogenous mitochondria.

In some embodiments, the protein of interest comprises an endogenous protein and/or a heterologous protein.

According to yet another aspect, the present disclosure provides an isolated cell or population of cells comprising isolated exogenous mitochondria substantially free of mitochondrial DNA (mtDNA) (Rho0-mito) and overexpressing a protein of interest, wherein the cell or population of cells comprising the isolated exogenous can synthesize the protein of interest at an improved synthesis rate/level, compared to the synthesis rate/level of the protein in a cell not comprising isolated exogenous mitochondria.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures.

FIG. 1A shows representative microscope images of cells presenting an increase in protein synthesis in LCL cells derived from a Pearson syndrome patient that were treated/contacted with mitochondria isolated from placenta (treated LCL cells), relative to non-treated Pearson LCL cells (non-treated LCL cells). The isolated mitochondria contain their mtDNA.

FIGS. 1B-1C are graphs presenting quantification of the increase in protein synthesis rate/level in the LCL cells derived from the Pearson syndrome patient Pearson syndrome of FIG. 1A (FIG. 1B) and quantification of an increase in protein synthesis rate/level in LCL cells derived from a Leigh syndrome patient that were treated with mitochondria isolated from placenta—Leigh syndrome (FIG. 1C), compared to non-treated cells.

FIG. 2A shows representative microscope images of LCL cells derived from a Pearson syndrome patient presenting a dose dependent increase in protein synthesis in cells treated/contacted with high number of mitochondria with respect to cells treated with low number of mitochondria and non-treated cells. The isolated mitochondria contain their mtDNA.

FIGS. 2B-2C are graphs presenting quantification of the dose dependent increase in protein synthesis in the cells treated with high number of mitochondria with respect to the cells treated with low number of mitochondria and the non-treated cells previously shown in FIG. 2A.

FIG. 3A is a graph illustrating a decrease in levels of branched chain amino acids (BCAA) in LCL cells incubated with isolated mitochondria derived from Rho0 cells, as measured by ELISA. Shown are BCAA levels in LCL cells from an MSUD patient incubated with Rho0 mitochondria (MSDU+mito) compared to levels in LCL cells from an MSUD patient without incubation with Rho0 mitochondria (MSDU) and to normal LCL cells isolated from a healthy donor (positive control (PS)). MSUD-mito were incubated with 4.4 mU CS activity of Rho0 mitochondria per 1 million cells and demonstrate roughly 13% decrease in cellular BCAA levels, according to some embodiments.

FIG. 3B is a graph illustrating an increase in levels of protein synthesis in LCL cells incubated with isolated mitochondria derived from Rho0 cells, as measured by incorporation of puromycin with a tyrosyl t-RNA analog. Shown are mean fluorescence levels of anti-puromycin in LCL cells from an MSUD patient incubated with isolated mitochondria derived from Rho0 cells (MSDU+mito) compared to levels in LCL cells from an MSUD patient without incubation with Rho0 mitochondria (MSDU) and to normal LCL cells isolated from a healthy donor (positive control (PS)). MSUD+mito were incubated with 8.8 mU CS activity of Rho0 mitochondria per 1 million cells and demonstrate roughly 30% increase in protein synthesis levels 1 day after co-incubation, according to some embodiments.

FIG. 3C is a graph illustrating an increase in levels of intracellular ATP in LCL cells incubated with isolated mitochondria derived from Rho0 cells. Shown are ATP concentration in LCL cells from an MSUD patient cultured in either glucose (MSUD-Glu) or galactose (MSUD-Gal) medium for 8 days, compared with ATP concentration in corresponding cells previously underwent co-incubation with isolated mitochondria derived from Rho0 cells for 3 hours (MSUD-Glu+mito) and (MSUD-Gal+mito). MSUD-mito were incubated with 8.8 mU CS activity of Rho0 mitochondria per 1 million cells, according to some embodiments.

FIG. 3D is a graph illustrating a dose dependent increase in protein synthesis capability (median fluorescence intensity) per ATP content (nM) of LCL cells incubated with isolated mitochondria derived from Rho0 cells for 24 h. Shown is the Protein synthesis ability per ATP (PS/ATP) of untreated LCL cells from an MSUD patient (MSDU), LCL cells from an MSUD patient treated with low dose of Rho0-mitochondria (MSUD-low-mito) or high dose of Rho0-mitochondria (MSUD-high-mito), and LCL cells taken from a healthy patient (PC). MSUD-low mito were incubated with 2.2 mU CS activity of Rho0 mitochondria per 1 million cells; high mito were incubated with 8.8 mU CS activity of Rho0 mitochondria per 1 million cells, according to some embodiments.

FIG. 3E a graph illustrating a dose dependent increase in proliferation of LCL cells incubated with isolated mitochondria derived from Rho0 cells under conditions favoring glycolysis (glucose (Glu) supplemented media) or mitochondrial respiration (galactose (Gal) containing media), as measured 8 days after incubation with mitochondria isolated from Rho0 cells. Shown is the number of cells (cell count) of untreated LCL cells from an MSUD patient (MSDU), LCL cells from an MSUD patient treated with low dose of Rho0-mitochondria (MSUD-low-mito), or medium dose of Rho0-mitochondria (MSUD-med-mito), or high dose of Rho0-mitochondria (MSUD-high-mito). Proliferation in either media was improved after incubation with low, medium, or high Rho0-mitochondria doses (4.4, 8.8, or 17.6 mU CS activity per 1M cells, respectively), according to some embodiments.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure, however, it will also be apparent to one skilled in the art that the disclosure may be practiced without specific details being presented herein and that this invention is not limited to particular compositions, methods, and experimental conditions described, as such compositions, methods, and conditions may vary. It is also to be understood that the terminology used herein is for purposes of describing particular embodiments only, and is not intended to limit the scope of the present invention.

The present invention is based on the seminal discovery that isolated mtDNA-free mitochondria (Rho0-mito) are useful for transfer to cells to replace dysfunctional mitochondria or to deliver proteins, antibodies, nucleic acids, or small molecule therapeutics to a subject.

The invention is also based on the discovery that isolated exogenous mitochondria, and particularly isolated Rho0-mito can improve protein synthesis rate/level when in cells and therefore are useful for industrial protein production.

As used herein the term “mtDNA-free mitochondria” refers to isolated mitochondria substantially free of mitochondrial DNA (mtDNA). The terms “mtDNA-free mitochondria”, “mitochondria substantially free of mitochondrial DNA (mtDNA)” “Rho0” and “Rho0-mito” are herein used interchangeably.

As used herein, the term “isolated” may refer to the mitochondria of the present invention or cells comprising the same, and means either: 1) separated from at least some of the components with which it is usually associated in nature; 2) prepared or purified by a process that involves the hand of man; and/or 3) not occurring in nature.

The term “exogenous” is used herein to refer to the isolated mitochondria of the present invention being transferred to cells. In some instances, the terms “isolated” and “exogenous” may be herein used interchangeably to refer to the mitochondria of the present invention.

In accordance, in some embodiments, the exogenous mitochondria may refer to the isolated mitochondria that is transferred to cells in culture to improve the cell's protein production capability, or that the exogenous mitochondria may refer to the isolated mitochondria substantially free of mtDNA (Rho0-mito) for use as a therapeutic entity since mtDNA may be immunogenic. Each possibility is a separate embodiment.

As used herein, the terms “essentially” and “substantially” are synonymous and when referring to a stated material such as a composition, a substance, and the like, is meant to encompass variations of in some embodiments, ±0.1%, or in some embodiments, ±1%, or in some embodiments, ±2%, or in some embodiments, ±5% from a stated amount, as such variations/deviations are appropriate to perform the disclosed methods. According to some embodiments, the terms “essentially devoid of” or “substantially free of” are synonymous and may refer to a stated material as either entirely absent or present in a residual amount, such as less than 5%, or less than 2%, or less than 1%, or less than 0.1% are present with respect to the initial amount of the stated material or with respect to the total % of all other components. Each possibility is a separate embodiment.

In some embodiments, the isolated mitochondria substantially free of mitochondrial DNA (mtDNA) (Rho0-mito) comprises less than 5%, or less than 2%, or less than 1%, or less than 0.1% mitochondrial DNA (mtDNA) with respect to the total % of mtDNA in the isolated mitochondria composition or with respect to the initial amount of mtDNA before isolation from the Rho0-cells.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” or “approx.” when referring to a measurable value such as an amount, a temporal duration, and the like, is meant to encompass deviations/variations of ±20% or in some embodiments ±10%, or in some embodiments ±5%, or in some embodiments ±1%, or in some embodiments ±0.1% from the specified value, as such deviations are appropriate to perform the disclosed methods. Each possibility is a separate embodiment. As used herein, the term “comprising” is synonymous with the terms “including,” “containing,” or “characterized by,” and is inclusive or open-ended i.e. does not exclude additional, unrecited elements. According to some embodiments, the term comprising may be replaced with the term with the term “consisting of” which excludes any element, step, or ingredient not specified in the claim. According to some embodiments, the term comprising may be replaced with the term “consisting essentially of” which limits the scope of a claim to the specified materials or steps “and those that do not materially affect the basic and novel characteristics” of the claimed invention.

As used herein, the terms “prevent”, “reduce”, “attenuate”, “ameliorate”, “alleviate”, and “inhibit” may be used interchangeably.

As used herein, the terms “enhanced”, “increased”, “improved” “elevated” may be used interchangeably.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The terms “subject”, “patient”, or “subjects” as used herein, refer to a refer to a human or other mammal, including ungulates, rodents, or primates; for example, horses, cattle, sheep, pigs, goats, llamas, camels, dogs, cats, birds, ferrets, rabbits, squirrels, mice, opossums, lemurs, or rats. In one embodiment, the subject is a human subject.

The term “effective amount” refers to the amount of a compound, composition, or formulation that is sufficient to treat a condition or disease, to produce desirable effects or results, or to reduce, ease, or arrest symptoms of a condition or disease. “Effective amount” is used interchangeably with the term “therapeutically effective amount”.

The terms “treatment” and “treating” as used herein, refer to a prophylactic or preventative measure having a therapeutic effect and slowing down, or at least partially alleviating or abrogating an undesirable condition in the organism of a subject. Those in need of treatment include those already with the condition as well as those prone to having the condition or those in whom the condition is to be prevented (prophylaxis). Generally, a treatment reduces, stabilizes, or inhibits progression of a symptom that is associated with the presence and/or progression of an undesirable condition. The term “therapeutic effect” refers to the inhibition or activation of factors causing or contributing to the undesirable condition. A therapeutic effect relieves to some extent one or more of the symptoms of an undesirable condition or disease. The term “undesirable condition” refers to a function in the cells or tissues of an organism that deviates from the optimal functions in that organism.

As used herein, the term “administer” refers to any way a treatment is given to a patient or subject. “Administer” refers to any mode of transferring, delivering, introducing, or transporting matter such as a compound, e.g. a pharmaceutical compound, or other agent such as an antisense oligonucleotide, to a subject. Administering can be accomplished via topical, intravenous, intramuscular, systemic, oral, or parenteral methods.

As used herein, the term “composition”, as used herein, refers to a composition including mitochondria. According to some embodiments, the term “the composition”, as used herein, refers to mitochondria. According to some embodiments, the composition of the invention includes partially purified mitochondria. According to some embodiments, the composition of the invention includes isolated mitochondria. According to other embodiments, the composition of the invention includes at least one of isolated mitochondria, partially purified mitochondria, and a combination thereof.

In certain embodiments, the mitochondria are frozen. In certain embodiments, the mitochondria are thawed. The term “thawed” as used herein means have undergone at least one freeze-thaw cycle, e.g. were at least once frozen but now un-frozen.

In certain embodiments, the composition includes about 5 μg/ml to about 90 μg/ml of mitochondria, about 5 μg/ml to about 80 μg/ml of mitochondria, about 5 μg/ml to about 70 μg/ml of mitochondria, about 5 μg/ml to about 60 μg/ml of mitochondria, about 5 μg/ml to about 50 μg/ml of mitochondria or about 5 μg/ml to about 30 μg/ml of mitochondria. Each possibility is a separate embodiment. In certain embodiments, the composition includes about 12.5 μg/ml of mitochondria.

According to some embodiments, the total concentration of the mitochondria in the composition of the invention is between about 10-20 μg/ml. According to other embodiments, the concentration is between about 10-15 μg/ml. According to some embodiments, the concentration is about 12.5 μg/ml. According to some embodiments, the concentration is at least about 10 μg/ml. According to some embodiments, the concentration is at least about 12.5 μg/ml. According to some embodiments, the concentration is no more than about 50 μg/ml. According to some embodiments, the concentration is between about 1-50 μg/ml. According to some embodiments, the concentration is between about 5-50 μg/ml. According to some embodiments, the concentration is between about 0.1-50 μg/ml. According to some embodiments, the concentration is no more than about 100 μg/ml.

In certain embodiments, the composition includes at least about 1 μg/ml of mitochondria, at least about 3 μg/ml of mitochondria, at least about 5 μg/ml of mitochondria, at least about 10 μg/ml of mitochondria, at least about 20 μg/ml of mitochondria, at least about 30 μg/ml of mitochondria, at least about 40 μg/ml of mitochondria, or at least about 50 μg/ml of mitochondria. Each possibility is a separate embodiment.

In certain embodiments, the composition is frozen. In certain embodiments, the composition is frozen at 0° C. or at a lower temperature. In certain embodiments, the composition is frozen at −20° C. or at a lower temperature. In certain embodiments, the composition is frozen at −70° C. or at a lower temperature. In certain embodiments, the composition is frozen in liquid nitrogen.

In certain embodiments, the composition is thawed after being frozen. In certain embodiments, the composition is thawed and is at room temperature. In certain embodiments, the composition is thawed and is at a temperature of 15° C. to 30° C. In certain embodiments, the composition is thawed and is at 4° C. In certain embodiments, the composition is thawed and is at a temperature of about 30-37° C.

In certain embodiments, at least part of the composition includes mitochondria. In certain embodiments, at least 5% of the composition includes mitochondria. In certain embodiments, at least 10% of the composition includes mitochondria. In certain embodiments, at least 20% of the composition includes mitochondria. In certain embodiments, at least 40% of the composition includes mitochondria. In certain embodiments, at least 50% of the composition includes mitochondria and so on.

Transfer of protein/mRNA to a mitochondria before or after mtDNA depletion can be performed by various methods. For example, nucleic acid encoding a protein or mRNA with a target sequence to the mitochondria can be delivered via a vector. Vectors as described herein are delivered to the cell nucleus where expression (RNA/protein) can then be transported or transferred to the mitochondria. In some embodiments, the vector can be an integrating or non-integrating vector, referring to the ability of the vector to integrate the expression cassette and/or transgene into a genome of a cell. Either an integrating vector or a non-integrating vector can be used to deliver an expression cassette containing a gene operably linked to a regulatory element. In some embodiments, examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viruses have several advantages for delivery of nucleic acids, including high infectivity and/or tropism for certain target cells or tissues. Each possibility is a separate embodiment. In some cases, a virus is used to deliver a nucleic acid molecule or expression cassette including one or more regulatory elements, as described herein, operably linked to a gene. (See also Faria et al., Pharmaceutics. 2023 February; 15(2): 572, herein incorporated by reference).

Expression vectors can include regulatory elements to control transcription of the polynucleotide of interest. Non-limiting examples of regulatory elements include promoter, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Vectors also can include other elements. For example, a vector can include a nucleic acid that encodes a signal peptide such that the encoded polypeptide is directed to a particular cellular location (e.g., a signal sequence targeting a protein to the mitochondria) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphotransferase, thymidine kinase (TK), and xanthin-guanine phosphoribosyl transferase (XGPRT). Such markers are useful for selecting stable transformants in culture. Regulatory sequences can generally be derived from mammalian, microbial, viral or insect genes. The ability to replicate in a host, usually conferred by an origin of replication, and a selection gene to facilitate recognition of transformants. Those of skill in the art can select a suitable regulatory region to be included in such a vector.

According to some embodiments, a vector can be a genomic integrated vector, or “integrated vector,” which can become integrated into the chromosomal DNA of the host cell; or an episomal vector, e.g., a nucleic acid capable of extra-chromosomal replication. Each possibility is a separate embodiment. Vectors capable of directing the expression of genes to which they are operatively linked are referred to herein as “expression vectors”. In some embodiments, viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Each possibility is a separate embodiment. Viral vectors are modified so the native tropism and pathogenicity of the virus has been altered or removed. The genome of a virus also can be modified to increase its infectivity and to accommodate packaging of the nucleic acid encoding the polypeptide of interest.

The term “AAV” is an abbreviation for adeno-associated virus, and can be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAV5, AAV6, AAV7, AAV8, AAV9, AAV10, AAV11, AAV 12, rhlO, and hybrids thereof, avian AAV, bovine AAV, canine AAV, equine AAV, primate AAV, non-primate AAV, and ovine AAV.

The use of “lentiviral vector” in gene therapy refers to a method by which genes can be inserted, modified, or deleted in organisms using lentivirus. Lentivirus are a family of viruses which infect by inserting DNA into their host cells' genome. Many such viruses have been the basis of research using viruses in gene therapy, but the lentivirus is unique in its ability to infect non-dividing cells, and therefore has a wider range of potential applications. Lentiviruses can become endogenous (ERV), integrating their genome into the host germline genome, so that the virus is henceforth inherited by the host's descendants. To be effective in gene therapy, there must be insertion, alteration and/or removal of host cell genes. To do this, scientists use the lentivirus' mechanisms of infection to achieve a desired outcome to gene therapy. In some embodiments, non-limiting examples or lentivirus that can be used for gene therapy include those derived from bovine immunodeficiency virus, caprine arthritis encephalitis virus, equine infectious anemia virus, feline immunodeficiency virus, human immunodeficiency virus 1, human immunodeficiency virus 2, Jembrana disease virus, puma lentivirus, simian immunodeficiency virus or Visna-maedi virus. Each possibility is a separate embodiment.

In addition to the use of retro- and lentiviral-based vectors, vectors derived from other viruses, such as adenoviruses and adeno-associated viruses (AAV), may also be utilized for the modification of hematopoietic stem and progenitor cells.

As known to one of skill in the art, gene modification can be achieved by targeted gene editing by employing site-specific endonucleases to induce a double-stranded break (DSB) in the DNA (e.g., zinc finger nucleases (ZFNs), Transcription activator like effector nucleases (TALENs), clustered regularly interspaced short palindromic repeats (CRISPR).

The term “therapeutically acceptable” refers to those agents, or a derivative thereof, which are suitable for use in contact with the tissues of patients without undue toxicity, irritation, and allergic response, are commensurate with a reasonable benefit/risk ratio, and are effective for their intended use.

The terms “treat,” “treated,” “treating”, or “treatment” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total, whether induction of or maintenance of), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may also be preemptive in nature, i.e., it may include prevention of disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease and prolonging disease-free survival as compared to disease-free survival if not receiving treatment and prolonging disease-free survival as compared to disease-free survival if not receiving treatment.

The terms “expressing” and “expression” in reference to a polypeptide are intended to be understood in the ordinary meaning as used in the art. A polypeptide is expressed by a cell via transcription of a nucleic acid into mRNA, followed by translation into an initial polypeptide, which is folded and possibly further processed to a mature polypeptide. Hence, the statement that a cell or an organism is expressing such a polypeptide indicates that the polypeptide is found in or on the cell, and cells of the organism, respectively, and implies that the polypeptide has been synthesized by the expression machinery of the respective cell.

With regard to the respective biological process itself, the terms “expression”, “gene expression” or “expressing” refer to the entirety of regulatory pathways converting the information encoded in the nucleic acid sequence of a gene first into messenger RNA (mRNA) and then to a polypeptide. Accordingly, the expression of a gene includes its transcription into a primary pre-RNA, the processing of this pre-RNA into a mature RNA and the translation of the mRNA sequence into the corresponding amino acid sequence of the polypeptide. In this context, it is also noted that the term “gene product” refers not only to a polypeptide, including e.g., a mature polypeptide (including a splice variant thereof) encoded by that gene and a respective precursor protein where applicable, but also to the respective mRNA, which may be regarded as the “first gene product” during the course of gene expression.

The term “isolated” indicates that the cell, mitochondria, or the peptide or nucleic acid molecule has been removed from its normal physiological environment, e.g. a natural source, or that a peptide or nucleic acid is synthesized. Use of the term “isolated” indicates that a naturally occurring sequence has been removed from its normal cellular (i.e., chromosomal) environment. Thus, the sequence may be in a cell-free solution or placed in a different cellular environment. An isolated cell, isolated cells, or isolated mitochondria may for instance be included in a different medium such as an aqueous solution than provided originally or placed in a different physiological environment. Typically, isolated cells, mitochondria, peptides or nucleic acid molecule(s) constitute a higher fraction of the total cells, mitochondria, peptides or nucleic acid molecule(s) present in their environment, e.g., a solution or suspension as applicable, than in the environment from which they were taken.

Mitochondria can serve as particles to deliver cargo to target organs/cells, and more specifically they can enter cells and fuse to endogenous mitochondria, thus delivering the cargo to the desired place in the cell; for example, to mitochondria. Even without merging with endogenous networks, they can act as a sink for toxic intermediate metabolites; for example, if a missing enzyme is overexpressed in the exogenous mitochondria. In addition, mitochondria can after uptake be targeted to lysosomes to deliver cargo to these organelles.

Also, in any use of exogenous mitochondria as a therapeutic entity, mtDNA may be immunogenic. In any case where mitochondria are injected for medical use, mitochondria without mtDNA may be a safer solution. Advantageously if no mtDNA exists in the mitochondria, they can be used by direct injection and treat a broad range of diseases. Mitochondria free of mtDNA (Rho0) could be considered as an enzyme replacement therapy, where the mitochondria are the natural delivery vector of the enzyme.

In one embodiment, the present disclosure provided herein relates to an isolated mitochondria substantially free of mitochondria DNA (mtDNA); optionally such mitochondria include a heterologous protein or nucleic acid sequence. In one aspect, the heterologous nucleic acid sequence is single stranded or double stranded. In certain aspects, the nucleic acid sequence is in an expression vector. In various aspects, the heterologous protein or nucleic acid sequence includes a mitochondrial-targeting sequence. In one aspect, the nucleic acid sequence is DNA or RNA. In a further aspect, the protein is a peptide. In some aspects, the protein is an enzyme. In some aspects, the present disclosure relates to an isolated cell including mitochondria substantially free of mitochondria DNA (mtDNA)(Rho0-mito) and optionally the mitochondria include a heterologous protein or nucleic acid sequence.

Reference is made to Examples 1-2 describing a non-limiting example of production of isolated Rho0 mitochondria free of mtDNA, with or without overexpressing a protein or RNA of interest (mtDNA depleted mitochondria with heterologous protein or nucleic acid).

The term “heterologous” or “heterologous expression” as used herein should be broadly interpreted as referring to a protein or nucleic acid that is transferred into a cell that does not normally express that particular protein or nucleic acid or does not express that particular protein or nucleic acid sequence at normal levels, the heterologous protein or nucleic acid may be derived from a different organism or a different species in comparison to the organism or species the recipient mitochondria is isolated from, or that the heterologous protein or nucleic acid may be derived from a different cell type of the same species or organism, in comparison to the cell type the recipient mitochondria is isolated from. In some embodiments, the term “heterologous” refers to the fact that the transferred protein was initially cloned from a different cell type or a different species than the recipient mitochondria.

A non-limiting example for heterologous expression includes a heterologous protein or nucleic acid derived from human lung cell and transferred to recipient mitochondria isolated from human pancreatic cells.

Another non-limiting example includes overexpression of an enzyme that is mutated or has partially or completely lost its function in the target cells.

Another non limiting example includes overexpression of a protein that increases mitochondrial lipid metabolism for example UCP1 or UCP2, in some embodiments.

Another non-limiting example includes overexpression of a non-mitochondrial protein, with a mitochondrial targeting sequence, to be targeted to the mitochondria before isolation.

Another non-limiting example for heterologous expression includes a heterologous protein or nucleic acid derived from cells of a virus or plant cells, and is transferred to recipient mitochondria isolated from mammalian cells or different plant cells, respectively.

Reference is made to Example 3 describing heterologous expression of some proteins involved in mitochondria-related disease. These proteins of interest are produced with improved synthesis rate in cell cultures harboring exogenous mitochondria, or used as cargo in isolated Rho0 mitochondria for delivery in vivo.

In one aspect, the mitochondria include an active molecule or a chemical agent that elicits a pharmaceutical effect in a subject. In some embodiments, the active molecule may be an inorganic molecule, an organic molecule, a small organic molecule, a drug compound, a peptide, a polypeptide, such as an enzyme or transcription factor, an antibody, an antibody fragment, a peptidomimetic, a lipid, or a nucleic acid such as a DNA or RNA molecule. Each possibility is a separate embodiment. In another aspect, the mitochondria of the present disclosure includes a vaccine, a nutrient, an antibody, a therapeutic, or a reagent. Each possibility is a separate embodiment.

In one embodiment, the present disclosure provided herein relates to a method for producing the isolated mitochondria substantially free of mitochondria DNA (mtDNA) and optionally including a heterologous protein or nucleic acid sequence including: a) contacting a cell with a chemical agent (e.g, FCCP, Chlorfenapyr, Pinacyanol, Oligomycin or 2,4-dinitrophenol) or a heterologous nucleic acid encoding a mitochondrial targeting protein; b) removing mitochondrial DNA from the mitochondria; and c) isolating the mitochondria from the cell. In certain aspects, the nucleic acid sequence is an expression vector. In various aspects, the heterologous protein or nucleic acid sequence includes a mitochondrial-targeting sequence. In one aspect, the nucleic acid sequence is DNA or RNA. In a further aspect, the protein is a peptide. In some aspects, the protein is an enzyme.

The term “chemical agent” as used herein may refer to a compound that enters/transfers to mitochondria and has a therapeutic effect on a subject, for example a compound that has an anti-cancerous activity or that enhances mitochondria function. In some embodiments a chemical agent may include a drug or small molecule that is loaded into the mitochondria for delivery. For example, to enhance mitochondria function or fight cancer. In some embodiments, non-limiting examples of chemical agents include, but is not limited to a compound that that has anti-cancerous activity such as: FCCP, Chlorfenapyr, Pinacyanol, and Oligomycin or 2,4-dinitrophenol. In some embodiments, non-limiting examples of chemical agents include, but is not limited to a compound that enhances mitochondrial function such as: Coenzyme Q10, alpha lipoic acid, resveratrol.

In some embodiments, the chemical agent comprises FCCP, Chlorfenapyr, Pinacyanol, and Oligomycin or 2,4-dinitrophenol, Coenzyme Q10, alpha lipoic acid, and resveratrol.

In another embodiment, the techniques described herein relate to a method for treating a disease or disorder in a subject, including administering to the subject an effective amount of an isolated mitochondria substantially free of mitochondria DNA (mtDNA) or optionally including a heterologous protein or nucleic acid sequence. In one aspect, administering the mitochondria delivers an effective amount of an active molecule that elicits a pharmaceutical effect in a subject. The active molecule may be an inorganic molecule, an organic molecule, a small organic molecule, a drug compound, a peptide, a polypeptide, such as an enzyme or transcription factor, an antibody, an antibody fragment, a peptidomimetic, a lipid, or a nucleic acid such as a DNA or RNA molecule. In another aspect, the mitochondria of the present disclosure delivers an effective amount of a vaccine, a nutrient, an antibody, a therapeutic, or a reagent.

As used herein, “optional” or “optionally” means that the subsequently described event or circumstance does or does not occur, and that the description includes instances where said event or circumstance occurs and instances where it does not.

In one aspect, the disease or disorder is selected from maple syrup syndrome, citrin deficiency, Nieman-Pick disease, X-linked adrenoleukodystrophy (X-ALD), Pyruvate dehydrogenase deficiency, respiratory chain diseases, mutations in mitochondrial respiratory chain proteins, and fatty acid metabolism disorders, or any combination thereof. In some embodiments, the disease or disorder comprises obesity or cancer. In some embodiments, the disease or disorder comprises cancer.

In one aspect, the disease or disorder is selected from maple syrup syndrome, citrin deficiency, Nieman-Pick disease, X-linked adrenoleukodystrophy (X-ALD), Pyruvate dehydrogenase deficiency, respiratory chain diseases, mutations in mitochondrial respiratory chain proteins, fatty acid metabolism disorders, obesity and cancer, or any combination thereof.

In one aspect, the isolated mitochondria substantially free of mitochondria DNA (mtDNA) including a heterologous protein or nucleic acid sequence is cryogenically preserved.

To create mtDNA-free mitochondria, cells are grown in culture and mtDNA-free cells (e.g., Rho0) are produced using ethidium bromide or any other method known in the art (for example, 4-Thio-dt or ECORI). Ref: Bai et al., 2021, Fernández-Moreno et al., 2016, Alexeyev et al., 2018 (other options described in: Kukat et al., 2008, McLaughlin et al., 2021).

Mitochondria are isolated from the cultured cell and qualified as to number of mitochondria, functional status, and lack of mtDNA, then optionally cryopreserved.

To over-express or target cargo to the mitochondria, prior to extracting the mitochondria, a mitochondrial or non-mitochondrial RNA, protein, peptide, or enzyme is produced in the mitochondria or in the cytoplasm and targeted to the mitochondria, prior to isolation and cryopreservation.

Mitochondria are used to treat different diseases caused by lack of mitochondrial function or by lack of or mutated mitochondrial enzymes or proteins. Cells can be preserved prior to isolation of mitochondria, either with or without overexpression of a mitochondrially-targeted cargo. The isolated mitochondria can be cryopreserved as well, either with or without mitochondrially-targeted cargo.

In some embodiments, administration of mitochondria can be local or systemic, though intramuscular, intro-ocular, dermal, into the heart, intracerebroventricular (ICV), intrathecal (IT), intravenous (IV), or through intravascular (IV) or intraperitoneal (IP) injection. Each possibility is a separate embodiment. Administration can also be through inhalation or nasal spray. For IV administration, the liver and lungs are a possible target. The bloodstream is another possible target.

The present composition can be used for therapeutic treatment of diseases including maple syrup syndrome, citrin deficiency, Nieman-Pick disease, X-linked adrenoleukodystrophy (X-ALD), Pyruvate dehydrogenase deficiency, respiratory chain diseases, mutations in mitochondrial respiratory chain proteins, or fatty acid metabolism disorders. Each possibility is a separate embodiment. In some embodiments, the mitochondria of the present disclosure can also be used to deliver vaccines, nutrients, antibodies, therapeutics, or reagents. Each possibility is a separate embodiment.

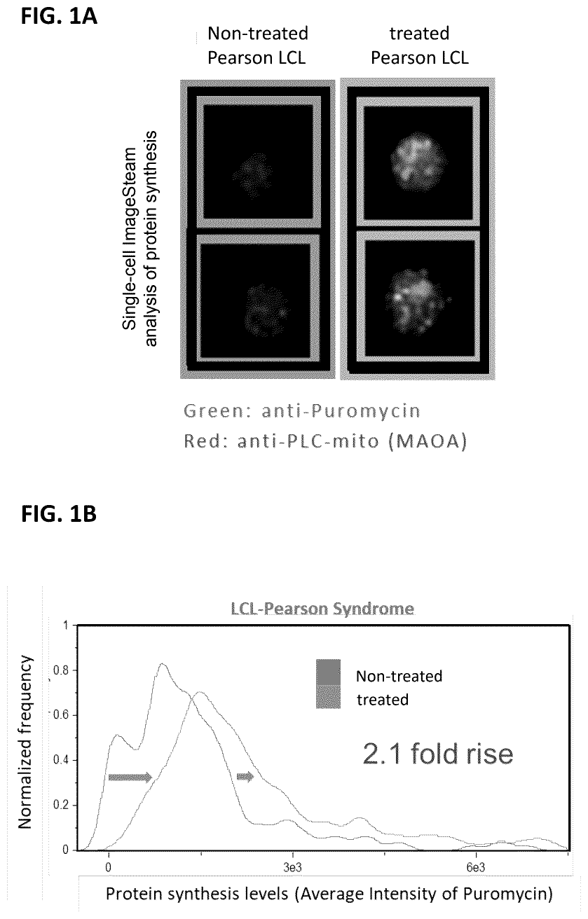

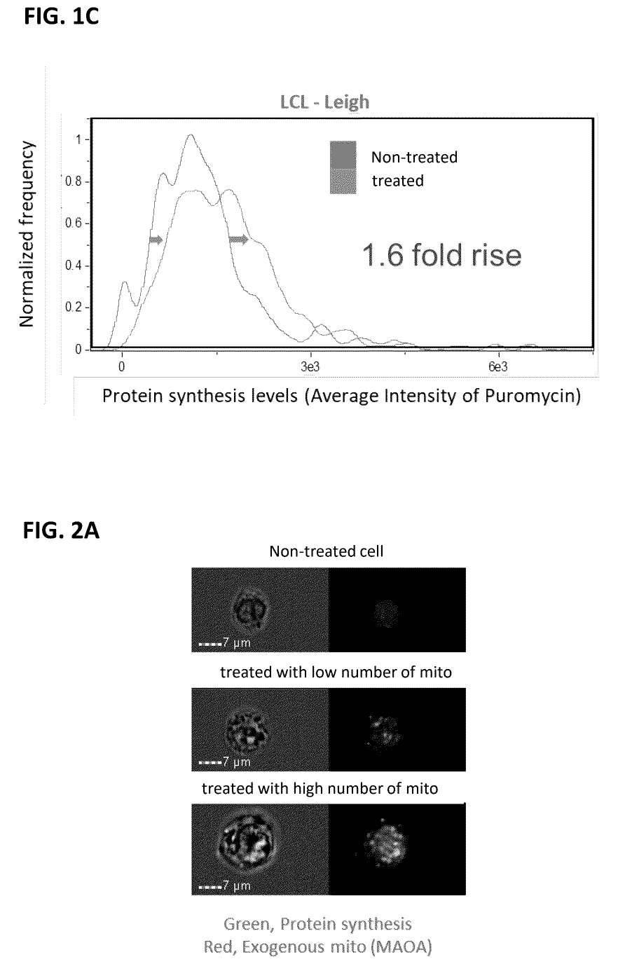

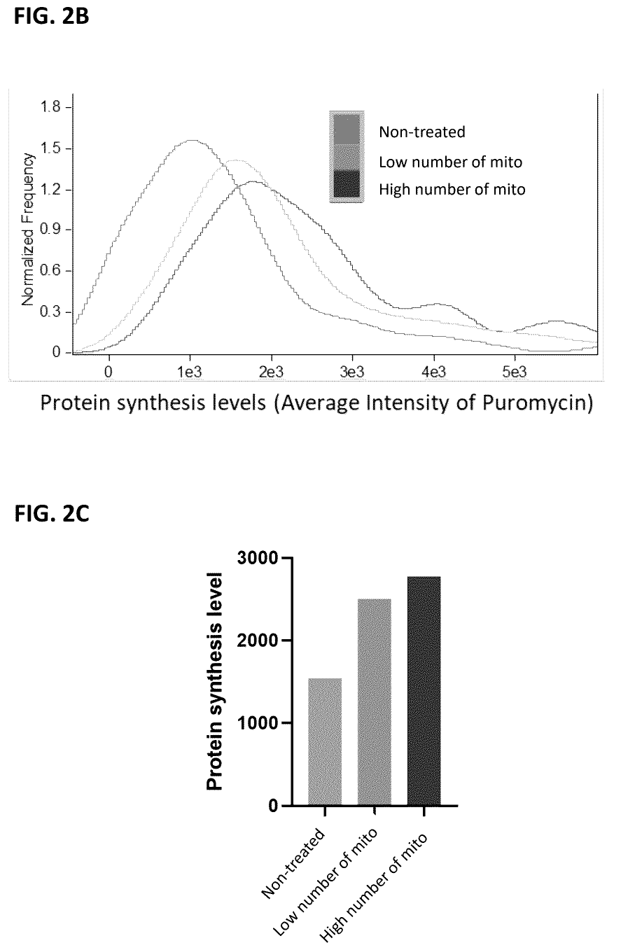

According to some aspects the disclosure provides a method for increasing protein synthesis in a cell, the method comprises: expressing a protein of interest in a cell in culture and contacting the cell with isolated exogenous mitochondria; thereby enhancing synthesis rate/level of a protein expressed in the cell containing the exogenous mitochondria. Reference is made to Example 5, FIGS. 1A-1C, and FIGS. 2A-2C.

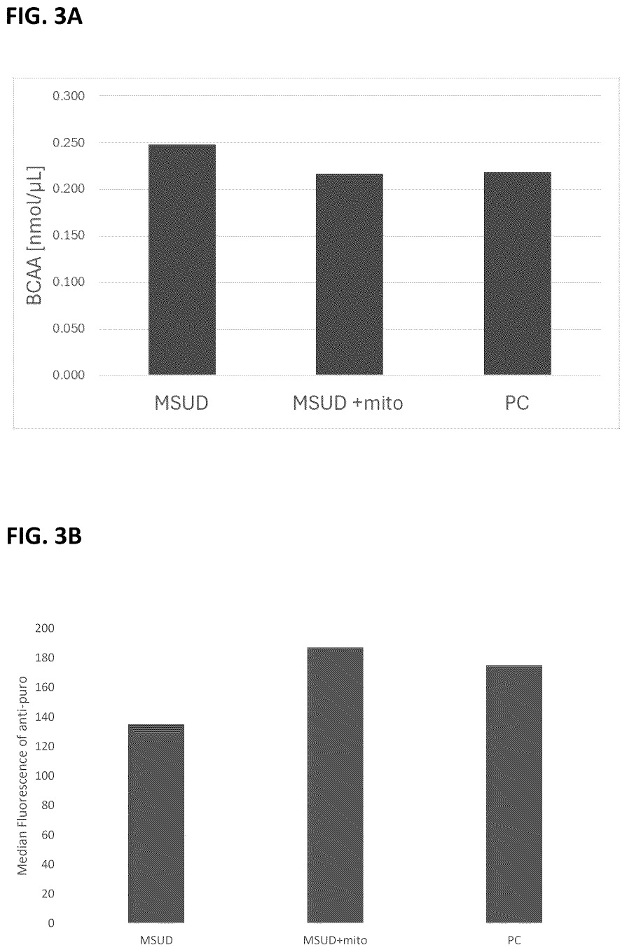

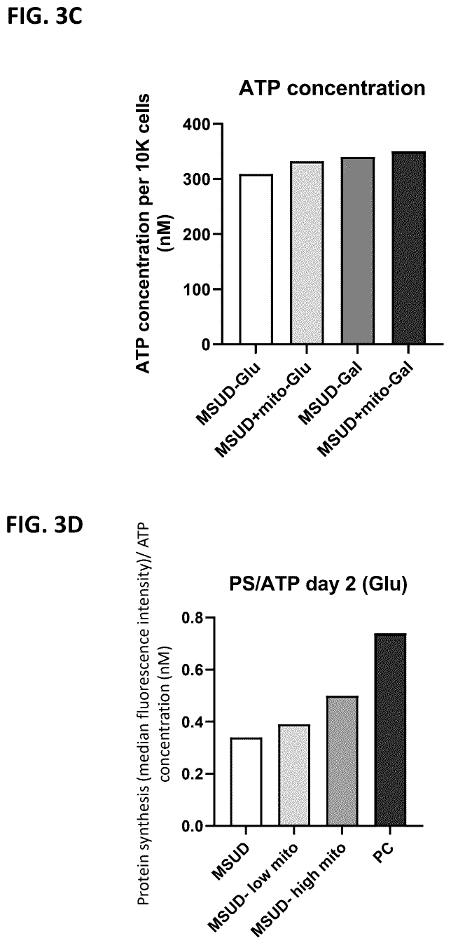

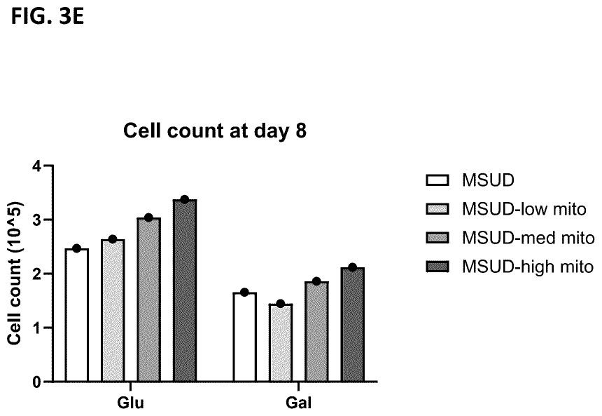

According to some aspects the disclosure provides a method for increasing protein synthesis in a cell, the method comprises: expressing a protein of interest in a cell in culture and contacting the cell with isolated exogenous mitochondria substantially free of mitochondrial DNA (mtDNA)(Rho0); thereby enhancing synthesis rate/level of a protein expressed in the cell containing the exogenous mitochondria. Reference is made to Example 6, FIGS. 3A-3E.

In some embodiments, the protein of interest comprises an endogenous protein and/or a heterologous protein.

In some embodiments, the method further comprising isolating the protein of interest from the cells or culture.

Presented below are examples concerning compositions and methods of producing mitochondria substantially free of mtDNA, with or without being loaded with cargo for targeted cargo delivery to cells of a subject in need, contemplated for the discussed applications, as well as to methods for increasing protein synthesis in a cell. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1: Creation of Rho0-Mito (mtDNA Depleted Mitochondria)

1. HeLa cells are grown in culture medium (DMEM, 10% FCS) with 0.1-2 μg/mL Ethidium Bromide for up to 30 days, with or without 50-200 μM 2′3′dideoxycytidine (ddC);
2. DNA is extracted from the cells and mtDNA copies are measured using qPCR. The DNA is also sequenced to demonstrate reduction of mtDNA content relative to genomic content;
3. Cells are grown in the presence of pyruvate and uridine;
4. Mitochondria are isolated from the cultured cells using sucrose buffer and cryopreserved;
5. Fresh or cryopreserved mitochondria (after thawing) are incubated with diseased cells and function is measured (as per examples below), according to some embodiments.

This example is a non-limiting example for production of isolated mitochondria substantially free of mtDNA. In some embodiments, alternative reagents are used, for example, Ethidium Bromide is replaced with another treatment known in the art to eliminate mtDNA, and in some embodiments, other cell types than Hela cells are used as donors of mitochondria. In some embodiments, the order of some steps may be different, or some steps may be omitted.

### Example 2: Creation of Rho0-Mito Overexpressing a Protein or RNA of Interest (mtDNA Depleted Mitochondria with Heterologous Protein or Nucleic Acid)

1. HeLa cells are grown in culture medium (DMEM, 10% FCS) with 0.1-2 μg/mL Ethidium Bromide for up to 30 days, with or without 50-200 μM 2′3′dideoxycytidine (ddC);
2. DNA is extracted from the cells and mtDNA copies are measured using qPCR. The DNA is also sequenced to demonstrate reduction of mtDNA content relative to genomic content;
3. Cells are grown in the presence of pyruvate and uridine;
4. Cells are transfected with expression vector to over-express target protein/peptide/enzyme/RNA. Expression vector can be codon-optimized sequence or a shorter functional version of the gene. Expression vector can also include a MTS attached to the gene for mitochondrial targeting of the overexpressed protein;
5. Mitochondria are isolated from the cultured cells using sucrose buffer and cryopreserved;
6. Fresh or cryopreserved mitochondria (after thawing) are incubated with diseased cells and function is measured (as per examples below), according to some embodiments.

This example is a non-limiting example for production of isolated mitochondria substantially free of mtDNA overexpressing a protein or RNA of interest. In some embodiments, alternative reagents are used, and the order of the steps is changed, for example, Ethidium Bromide is replaced with another treatment known in the art to eliminate mtDNA, or for example, the step of transfection with expression vector to over-express target proteins proceed the step of mtDNA removal, or for example, other cell types than Hela cells are used as donors of mitochondria. In some embodiments, the order of some steps may be different, or some steps may be omitted.

### Example 3: Improvement in Protein Synthesis and Function after Treatment of Cells with Rho0-Mito

1. In general assessments are performed on cells harboring the different mutations. These can be patient-derived cells (for example, fibroblasts) or LCL lines (for example those created by transformation of patient-derived cells), in some embodiment.
2. Maple Syrup disease cells carrying mutations in the BCKDH complex are grown in culture and treated with mtDNA-free mitochondria with or without over-expression of the normal BCKDH protein, in some embodiment. The levels of Leucin, Isoleucine and Valine are measured in the medium at 6 h, 12 h, 24 h and 48 h after incubation with mitochondria.
3. Pyruvate Dehydrogenase complex deficiency cells are grown in culture and treated with mtDNA-free mitochondria with or without over expression of the normal protein, in some embodiment. The levels of lactate are measured in the medium at 6 h, 12 h, 24 h and 48 h after incubation with mitochondria.
4. Mitochondrial DNA depleted cells, carrying a mutation in TFAM or POLG, are grown in culture and treated with mtDNA-free mitochondria with or without over-expression of the normal protein, in some embodiment. The number of mtDNA copy numbers are measured in the cells at 24 and 48 h after incubation with mitochondria.
5. TK/DGUOK mutation cells, carrying a mutation in TK or DGUOK gene, are grown in culture and treated with mtDNA-free mitochondria with or without over-expression of the normal protein, in some embodiment. The levels of enzymatic activity are measured in the cells at 24 and 48 h after incubation with mitochondria.
6. Cells carrying a mutation in Nieman Pick gene SPMD1, are grown in culture and treated with mtDNA-free mitochondria with over-expression of the normal protein with an MTS sequence, in some embodiment. The levels of SMPD1 protein is measured in the cells at 24 and 48 h after incubation with mitochondria.
7. Citrin deficiency cells (from CTLN2 patients), carrying a mutation in SLC25A13, are grown in culture and treated with mtDNA-free mitochondria with over-expression of the normal protein with an MTS sequence, in some embodiment. The levels of SMPD1 protein is measured in the cells at 24 and 48 h after incubation with mitochondria.
8. Pyruvate dehydrogenase deficiency cells (from PDHA1 patients), carrying a mutation in PDHA1, are grown in culture and treated with mtDNA-free mitochondria with over-expression of the normal protein with an MTS sequence, in some embodiment. The levels of PDHA1 protein levels of lactic acid are measured in the cells at 24 and 48 h after incubation with mitochondria.
9. Cells carrying a mutation in one or more of: BCKDK, SLC25A13, SMPD1, ABCD1, SerpinA1, SerpinA3, CPT1A, SLC25A20, PDHA1, ACADVL, HADHA, ACADM, and ACADS, are grown in culture and treated with mtDNA-free mitochondria with over-expression of the normal protein with an MTS sequence, in some embodiment. Protein levels are measured in the cells at 24 and 48 h after incubation with mitochondria.

The aforementioned lists some non-limiting examples of proteins involved in mitochondria-related disease produced with improved synthesis rate in cells harboring exogenous mitochondria, or used as cargo for delivery in isolated Rho0 mitochondria.

### Example 4: Improvement in Polg mtDNA Copy Number and Liver Function after in Vivo Rho0-Mito Administration

1. Rho0-Mito are administered daily for a week to polg mice at age 4-8 months by IV administration.
2. Animals are sacrificed and peripheral blood and liver samples are harvested.
3. Mitochondrial content and function are measured on tissue samples.
   a. DNA is harvested from tissues and mtDNA copy number is measured by qPCR and by next generation Illumina sequencing to assess mtDNA mutation and deletion burden.
   b. Measures of mitochondrial function are performed on liver samples, assessing citrate synthase activity and succinate activity.
   c. Measures of liver function in blood (AST/ALT) are performed and compared with those from the same animals 1-2 weeks prior to treatment.

### Example 5: Protein Synthesis Rate Increases Post Mitochondria Treatment

LCL cells derived from a patient having Pearson syndrome were treated with isolated exogenous mitochondria to evaluate the effect of treatment on rate of proteins synthesis. As can be seen in FIG. 1A, single cell image analysis of immune-stained puromycin was indicative of an increase in protein synthesis as measured in treated Pearson LCL cells, relative to non-treated Pearson LCL cells.

As can be seen in FIG. 1B it was determined that protein synthesis rate/level in those mitochondria treated LCL cells—Pearson syndrome is 2.1-fold higher than the rate/level of non-treated LCL cells.

In addition, as can be seen in FIG. 1C, a similar 1.6-fold increase was detected in mitochondria treated LCL cells—derived from a patient having Leigh syndrome, compared to non-treated cells.

Next, as can be seen in FIG. 2A, a dose dependent increase in protein synthesis was observed in Pearson LCL cells treated with high number of mitochondria with respect to cells treated with low number of mitochondria and non-treated cells.

Quantification of the dose dependent increase is presented in FIGS. 2B-2C showing an increase of more than 75% in protein synthesis in cells treated with high number of mitochondria with respect to cells treated with low number of mitochondria and non-treated cells.

### Example 6: Improvement in Protein Synthesis and Proliferation after Treatment of Maple Syrup Urine Disease (MSUD) Cells with mtDNA-Free Mitochondria (Rho0-Mito)

Maple Syrup disease cells carrying mutations in the BCKDH complex were grown in culture and treated with mtDNA-free mitochondria (Rho0-mito) in order to evaluate the effect Rho0-mito exerts on the ability of MSUD cells to metabolize branched chain amino acids (BCAA), synthesis and express proteins, and proliferate.

### Materials and Methods

Cell lines—1) MSUD-LCL cell line is a commercially available cell line (Coriell Institute, GM01655) derived from a patient having Maple Syrup Urine Disease (MSUD), type IA. Branched-Chain Keto Acid Dehydrogenase E1, Alpha Polypeptide; BCKDHA. 2) Control-LCL cell line is a commercially available cell line (Coriell Institute, ND24284) derived from B-Lymphocytes of a healthy male donor. 3) A549p0 #1 cell line is a commercially available cell line (Kerafast, ESA113) Derived from A549 lung adenocarcinoma by treating cells with ethidium bromide. A549p0 #1a is a human cell line derived from lung adenocarcinoma that lacks mitochondrial DNA, lack functional mitochondrial electron transport chain, and is auxotrophic for uridine and pyruvate. The A549p0 #1 cell line is a non-limiting example of a Rho0 cell, according to some embodiment.

Growth of Rho-0 Cells—A549p0 #1 Rho-0 cells (A549p0 #1a) lack mitochondrial DNA were cultured in Dulbecco's Modified Eagle's Medium (DMEM) containing high glucose and 1 mM sodium pyruvate, supplemented with 10% FBS and 50 mg/L uridine. Cells were maintained at 37° C. in a humidified incubator with 5% CO₂. Media were replaced every 2-3 days, and cells were passaged at 70-80% confluency.

Growth of LCL Cells—LCL-MSUD cells from a Maple Syrup Urine Disease patient were grown in RPMI-1640 medium supplemented with 20% FBS. LCL-Control cells were grown in RPMI-1640 medium supplemented with 15% FBS. Both cell lines were incubated at 37° C. in 5% CO₂, with medium changes every 2-3 days.

Mitochondria isolation from Rho-0 Cells—Mitochondria were isolated from Rho-0 cells using a modified differential centrifugation method. Rho-0 cells were harvested by trypsinization, washed twice in cold PBS, and the cell pellet was flash-frozen on dry ice and stored at −80° C. for 5 minutes. After freezing, the cell pellet was resuspended in cold sucrose solution and homogenized using a Dounce homogenizer. The homogenate was centrifuged at 1,000×g for 10 minutes at 4° C. The supernatant was collected and centrifuged again at 1,000×g for 10 minutes at 4° C. to remove residual cell debris and unbroken cells. The resulting supernatant was then centrifuged at 8,000×g for 15 minutes at 4° C. to pellet the mitochondrial fraction. The mitochondrial pellet was resuspended in cold sucrose solution for cryopreservation and subsequent use.

Co-incubation of Rho-0 Mitochondria with LCL-MSUD Cells—Isolated mitochondria from Rho-0 cells were thawed and co-incubated with LCL-MSUD cells to assess functional complementation. LCL-MSUD cells were seeded in a 24-well plate at a density of 0.5×10⁶ cells per well and incubated for 3 hours with Rho-0 mitochondria at varying concentrations, based on citrate synthase (CS) activity, as follows: 0, 2.2, 4.4, 8.8, and 17.6 Units/ml. After co-incubation, the cells were washed twice with PBS to remove unincorporated mitochondria and prepared for subsequent readout assays. LCL-control was used as a control LCL cell line. It was seeded in a separate set of wells under the same conditions (0.5×10⁶ cells per well, 3-hour incubation), but without the addition of Rho-0 mitochondria.

Seahorse Assay (Readout Assay)—Mitochondrial function in co-incubated LCL-MSUD cells was evaluated using the Seahorse XFe8 analyzer in an 8-well format. Oxygen consumption rates (OCR) were measured to assess basal respiration and ATP production. Cells were seeded in Seahorse XF8 plates and treated with oligomycin (1 μM) to measure ATP-linked respiration, followed by baseline OCR measurements.

Branched-Chain Amino Acids (BCAA) Kit (Readout Assay)—Branched-chain amino acid levels were measured using a commercially available BCAA assay kit following the manufacturer's protocol. Co-incubated cells were lysed, and their supernatants were used for quantifying BCAA concentrations by colorimetric analysis at 450 nm.

Growth on Galactose vs. Glucose Medium (Readout Assay)—To assess mitochondrial functionality post-co-incubation, LCL-MSUD cells were seeded in duplicates into 6-well plates at a density of 0.5×10⁶ cells per well and cultured in RPMI-1640 medium without glucose, supplemented with either 10 mM galactose or 25 mM glucose, and 20% FBS. Cell counts were taken at 1, 3, and 7 days after co-incubation to track growth in both conditions. Counting was performed using an automated cell counter to compare growth in galactose (requiring mitochondrial respiration) and glucose (supporting glycolysis).

Protein Synthesis Assay with Puromycin (Readout Assay)—Protein synthesis rates were evaluated using puromycin incorporation. After co-incubation, cells were treated with 1 μg/mL puromycin for 30 minutes. Following puromycin treatment, cells were harvested and analyzed by flow cytometry (FACS) to quantify the incorporation of puromycin into nascent polypeptides, reflecting protein synthesis levels. Anti-Puromycin antibody Alexa Fluor 647 Conjugate Antibody was used for the FACS analysis.

### Results

Initially, the ability of MSUD LCL cells to metabolize branched chain amino acids (BCAA) was evaluated in cells from an MSUD patient incubated with or without isolated mitochondria derived from Rho0 cells—Rho0 mitochondria (Rho0-mito)—and compared to BCAA levels in normal LCL cells (positive control (PC) isolated from a healthy donor).

As can be seen in FIG. 3A MSUD LCL Rho0-mito cells (incubated with 4.4 mU CS activity of Rho0 mitochondria per 1 million cells) demonstrated about 13% decrease in cellular BCAA levels, as measured by ELISA compared to MSUD LCL that were not incubated with Rho0-mito, bringing the levels of BCAA towards the normal levels detected in the PC, according to some embodiments.

Next, the protein synthesis capability of the MSUD LCL cells was assessed in the same setting wherein the MSUD LCL cells were incubated with or without Rho0 mitochondria (Rho0-mito) and the level of protein synthesis was compared to levels in normal LCL cells (positive control (PC) isolated from a healthy donor).

As can be seen in FIG. 3B MSUD LCL Rho0-mito cells ) incubated with 8.8 mU CS activity of Rho0 mitochondria per 1 million cells (demonstrated about 30% increase in protein synthesis levels 1 day after incubation with Rho0-mito, as measured by incorporation of puromycin with a tyrosyl t-RNA analog, compared to MSUD LCL that were not incubated with Rho0-mito, bringing the levels of protein synthesis about 5% above the normal levels detected in the PC, according to some embodiments.

Following the above, the intracellular ATP concentration of the LCL cells derived from the MSUD patient and cultured in either glucose (Glu) or galactose (Gal) medium for 8 days was measured. MSUD LCL cells underwent co-incubation with isolated mitochondria derived from Rho0 cells (MSUD LCL Rho0-mito) for 3 hours, and their intracellular ATP concentration was compared to that of MSUD LCL that were not incubated with Rho0-mito.

As can be seen in FIG. 3C, in each of the culture conditions, either glucose (Glu) or galactose (Gal), MSUD LCL Rho0-mito cells (co-incubated with 8.8 mU CS activity of Rho0 mitochondria per 1 million cells) exhibits up to 5-7% increase in ATP levels compared to MSUD LCL that were not co-incubated with Rho0-mito, according to some embodiments.

Furthermore, the ability of MSUD LCL cells to utilize ATP in protein synthesis (protein synthesis capability per ATP content) was measured and quantified 24 hours after co-incubation (in Glucose medium) with Rho0 mitochondria at ‘low’ and ‘high’ dosage (low or high MSUD LCL Rho0-mito), and their Protein synthesis ability (median fluorescence intensity) per ATP concentration (nM) was compared to that of untreated MSUD LCL cells that were not incubated with Rho0-mito and to that of normal LCL cells isolated from a healthy donor (positive control (PC)).

As can be seen in FIG. 3D, low-MSUD LCL Rho0-mito (incubated with 2.2 mU CS activity of Rho0 mitochondria per 1 million cells) and high-MSUD LCL Rho0-mito (incubated with 8.8 mU CS activity of Rho0 mitochondria per 1 million cells) demonstrated a gradual dose dependent increase of about 15% and about 40%, respectively, in the ability to utilize ATP to synthesize protein in comparison to untreated MSUD LCL thereby bringing the levels of protein synthesis per ATP (PS/ATP) towards the normal levels detected in the PC, according to some embodiments.

Lastly, the ability of MSUD LCL cells to proliferate in conditions favoring glycolysis (glucose supplemented media) or mitochondrial respiration (galactose containing media) was measured 8 days after co-incubation with mitochondria isolated from Rho0 cells at different dosage.

As can be seen in FIG. 3E, proliferation (cell count) in either Glu-or Gal-containing media was improved after co-incubation with low, medium, or high mitochondria doses (4.4, 8.8, or 17.6 mU CS activity per 1M cells, respectively). Proliferation of high-MSUD LCL Rho0-mito was increased by 30%-40% and about 20% in Glu media and Gal media, respectively, in comparison to untreated MSUD LCL cells that were not incubated with Rho0-mito, according to some embodiments.

While certain embodiments of the invention have been illustrated and described, it will be clear that the invention is not limited to the examples and embodiments described hereinabove. Numerous modifications, changes, variations, substitutions and equivalents will be apparent to those skilled in the art without departing from the spirit and scope of the present invention as described by the claims which follow.

1.-48. (canceled)

49. A method for treating a mitochondria-related disease or disorder in a subject, comprising administering to the subject an effective amount of a composition comprising isolated mitochondria substantially free of mitochondrial DNA (mtDNA).

50. The method of claim 49, wherein the disease or disorder is selected from the group consisting of maple syrup syndrome, citrin deficiency, Nieman-Pick disease, X-linked adrenoleukodystrophy (X-ALD), Pyruvate Dehydrogenase Deficiency, respiratory chain diseases, mutations in mitochondrial respiratory chain proteins, and fatty acid metabolism disorders, or any combination thereof.

51. The method of claim 49, wherein the isolated mitochondria comprise a heterologous protein, a nucleic acid sequence encoding a heterologous protein, and/or a chemical agent.

52. The method of claim 51, wherein the heterologous protein sequence comprises a mitochondria targeting signal.

53. The method of claim 51, wherein the heterologous protein is a peptide.

54. The method of claim 51, wherein the heterologous protein is an enzyme.

55. The method of claim 51, wherein the nucleic acid sequence encodes a gene selected from the group consisting of BCKDK, SLC25A13, SMPD1, ABCD1, SerpinA1, SerpinA3, CPT1A, SLC25A20, PDHA1, ACADVL, HADHA, ACADM, ACADS, BCKDH, TFAM, POLG, TK, DGUOK, and any combination thereof.

56. The method of claim 51, wherein the heterologous protein is selected from the group consisting of a branched-chain α-ketoacid dehydrogenase complex, citrin, acid sphingomyelinase, ATP binding cassette subfamily D member 1, alpha-1 antitrypsin, alpha 1-antichymotrypsin, carnitine palmitoyl transferase 1A, carnitine-acylcarnitine translocase, very long-chain specific acyl-CoA dehydrogenase, mitochondrial trifunctional protein, acyl-coenzyme A dehydrogenase, short-chain acyl-CoA dehydrogenase, and any combination thereof.

57. The method of claim 51, wherein the chemical agent is selected from the group consisting of FCCP, Chlorfenapyr, Pinacyanol, Oligomycin, 2,4-dinitrophenol, Coenzyme Q10, alpha lipoic acid, resveratrol, and any combination thereof.

58. A method for increasing protein synthesis in a cell, the method comprising contacting the cell with a composition comprising isolated mitochondria substantially free of mitochondrial DNA (mtDNA).

59. The method of claim 58, further comprising the following steps for preparing isolated mitochondria comprising a heterologous protein, prior to contacting the cell with the composition comprising the isolated mitochondria:
I. contacting a donor cell with a heterologous mitochondrially targeted protein or a nucleic acid encoding it;
II. removing mitochondrial DNA from the mitochondria of the donor cell to obtain a donor cell comprising mitochondria substantially free of mtDNA; and
III. isolating the mitochondria substantially free of mtDNA from the donor cell, wherein the first (contacting) step and the second (removing) step may be at any order, but before the third (isolating) step.

60. The method of claim 58, wherein contacting the cell with isolated exogenous mitochondria comprises incubating the cell in culture with 2.2 miliunits-17.6 miliunits CS activity of isolated exogenous mitochondria per 1.0E6 cells.

61. The method of claim 58, wherein the cell is a mammalian cell, a plant cell, or a yeast cell.

62. The method of claim 58, wherein the cell is a Chinese hamster ovary (CHO) cell, a CHO-GFP cell, a CHO Agarabi cell, a HeLa cell or a HEK293 cell.

63. The method of claim 58, wherein the protein synthesis level of a protein is increased by at least 1.2× compared to synthesis level of the protein in a cell not comprising the isolated exogenous mitochondria.

64. The method of claim 58, wherein the cell proliferates at a rate at least 1.2× compared to a cell not comprising the isolated exogenous mitochondria.
